(19) United States
(12) Patent Application Publication
(10) Pub. No.: US 2015/0102810 A1
(43) Pub. Date: Apr. 16, 2015

(54) METHOD AND APPARATUS TO CREATE AT LEAST ONE MAGNETIC RESONANCE IMAGE DATA SET

(71) Applicant: Siemens Aktiengesellschaft, Muenchen (DE)

(72) Inventors: David Grodzki, Erlangen (DE); Bjorn Heismann, Erlangen (DE)

(73) Assignee: Siemens Aktiengesellschaft, Muenchen (DE)

(21) Appl. No.: 14/511,609

(22) Filed: Oct. 10, 2014

(30) Foreign Application Priority Data
Oct. 10, 2013 (DE) 102013220465.5

(51) Int. Cl. G01R 33/56; A61B 5/145; G01R 33/385

(52) U.S. Cl. CPC G01R 33/5601; G01R 33/385; A61B 5/145
USPC 324/309; 324/322

(57) ABSTRACT

In a method and apparatus to create at least one magnetic resonance image data set in particular angiographic image data sets, first magnetic resonance image data are acquired using a first projection acquisition sequence, second magnetic resonance image data are acquired after administration of contrast agent, using a second projection acquisition sequence, and at least one magnetic resonance image data set is created using the first magnetic resonance image data and the second magnetic resonance image data.

### BACKGROUND OF THE INVENTION

1. Field of the Invention

The invention concerns a method to create at least one magnetic resonance image data set, a magnetic resonance apparatus, and a non-transitory, computer-readable data storage medium encoded with programming instruction, to implement such a method.

2. Description of the Prior Art

Angiography, in which blood vessels are depicted by diagnostic imaging methods, is a known medical diagnostic procedure. Magnetic resonance tomography enables a depiction of the blood vessels of an examined person by the use of contrast agent-assisted angiography sequences.

### SUMMARY OF THE INVENTION

An object of the invention is to provide an improved method to create angiographic image data sets by operation of a magnetic resonance apparatus.

The method to create at least one magnetic resonance image data set of an examination subject by operation of a magnetic resonance apparatus has the following steps.

First magnetic resonance image data are acquired using a first projection acquisition sequence.

Second magnetic resonance image data are acquired after administration of a contrast agent, using a second projection acquisition sequence.

At least one magnetic resonance image data set is created using the first magnetic resonance image data and the second magnetic resonance image data.

The contrast agent administration can include an introduction of a contrast agent into the examination subject. The introduction of the contrast agent into the examination subject can include an injection of the contrast agent, for example. The examination subject is preferably an examined person. The examined person can be a patient and/or a training person. The contrast agent can alternatively be introduced into the examination subject by an infusion pump, for example into a vein in the arm of the examined person.

Contrast agents are normally designed in order to cause particularly high or particularly low magnetic resonance signals. Contrast agents are used in order to depict vessels (in particular blood vessels) of an examined person that have contrast agent therein in magnetic resonance image data sets. Vessels having contrast agent are thereby vessels that supply blood that contains contrast agent. Normally, the blood having contrast agent (and therefore the inner volume of the vessels) is depicted in the magnetic resonance image data sets. The use of contrast agent is therefore particularly advantageous in angiography. Angiography typical includes the depiction of vessels—in particular blood vessels—by diagnostic imaging methods. The at least one magnetic resonance image data set is thus preferably an angiographic magnetic resonance image data set. The at least one magnetic resonance image data set will then typically depict vessels (in particular blood vessels) of an examined person.

Various contrast agents for contrasting blood are known to those skilled in the art, and need not be discussed in further detail herein. For example, the vessels having contrast agent are emphasized in that the intensity and/or amplitude of the magnetic resonance signals that are acquired from the vessels having contrast agent therein differing from the magnetic resonance signals of the surrounding tissue. The contrast agent can either intensify or attenuate the magnetic resonance signals. Such an attenuation or intensification depends on the selected magnetic resonance sequence, so that the first projection acquisition sequence and/or the second projection acquisition sequence preferably include imaging parameters that are designed for a depiction of the vessels having contrast agent. The use of these imaging parameters has the advantage that the vessels (for example arteries or veins) that have contrast agent-enhanced blood can be slightly differentiated from surrounding tissue.

The first magnetic resonance image data are typically acquired before the introduction of the contrast agent into the examination subject (i.e. the contrast agent administration). The acquisition of the first magnetic resonance image data is thus typically ended when the contrast agent is introduced into the examination subject. The first magnetic resonance image data can thus represent reference data that show the blood vessels of the examined person without contrast agent. The creation of the at least one magnetic resonance image data set can include the offsetting of the first magnetic resonance image data with the second magnetic resonance image data. The offsetting can be a subtraction (in particular a weighted subtraction) of the first magnetic resonance image data from the second magnetic resonance image data. The at least one magnetic resonance image data set can thus include an advantageous depiction of the contrast agent-carrying vessels of the examined person.

After the introduction of the contrast agent (i.e. the administration of the contrast agent), more than one set of second magnetic resonance image data can be acquired, such that multiple magnetic resonance image data sets can be created using the second magnetic resonance image data (sets). The multiple magnetic resonance image data sets can then describe a time curve of the contrast agent within the vessels of the examined person. For example, among these multiple magnetic resonance image data sets, one magnetic resonance image data set can show an arterial phase of the contrast agent enrichment. An additional magnetic resonance image data set of the multiple magnetic resonance image data sets can then show a venous phase of a propagation of the contrast agent, for example.

For example, magnetic resonance image data can be acquired by the application of phase coding gradients and frequency coding gradients to the magnetic resonance signals by the excitation of nuclear spins in the subject by the radiation of radio-frequency (RF) energy into the subject. The first magnetic resonance image data and the second magnetic resonance image data are typically only the raw data that represent the acquired magnetic resonance signals. The magnetic resonance image data are thus typically not directly available to an expert for diagnosis. Rather, at least one magnetic resonance image data set, which can be shown on a display unit and/or can be provided to an expert to create a diagnosis, is created using the first magnetic resonance image data and the second magnetic resonance image data.

Identical imaging parameters for the first projection acquisition sequence and the second projection acquisition sequence are used to acquire the first magnetic resonance image data and the second magnetic resonance image data. The field of view, a repetition time and an echo time between the first and second projection acquisition sequence are advantageously kept the same. Naturally, multiple projection acquisition sequences can be used (possibly with different imaging parameters, in particular projection directions) to acquire the first magnetic resonance image data and/or the second magnetic resonance image data.

A projection acquisition sequence typically foregoes coding of the acquired magnetic resonance signals in one spatial direction, in particular the slice direction. This spatial direction is then called a projection direction. For this purpose, a projection acquisition sequence includes no slice coding gradient along the spatial direction. A projection acquisition sequence along the projection direction consequently automatically transmits the acquired magnetic resonance signals already during the acquisition of the magnetic resonance signals. A projection acquisition sequence thus typically includes the acquisition of magnetic resonance signals with a slice thickness that goes to infinity in the projection direction and/or with a slice thickness that corresponds to the entire extent of the field of view of the projection acquisition sequence in the projection direction. Two-dimensional magnetic resonance image data are generated by a projection acquisition sequence. A projection acquisition sequence is comparable to radioscopy imaging in x-ray imaging, but the projection acquisition sequence is independently based on a completely different principle. The acquisition of the first magnetic resonance image data can be implemented using multiple first projection acquisition sequences. The acquisition of the second magnetic resonance image data can be implemented using multiple second projection acquisition sequences.

Specific methods to create angiographic magnetic resonance image data sets typically include three-dimensional magnetic resonance sequences, in particular magnetic resonance sequences with a coding in the slice direction. These magnetic resonance sequences present high demands on a gradient system since, for example, the acquisition of the magnetic resonance image data can be time-critical after the introduction of the contrast agent. In particular, high gradient strengths and/or high slew rates of the gradient system are required with regard to defined methods to create angiographic magnetic resonance image data sets. These magnetic resonance angiography measurements thus are typically very loud and cannot be implemented using weaker gradient systems, without performance loss.

The invention is based on the recognition that a slice coding is often not required in the creation of angiographic magnetic resonance image data sets and offers no particular advantages. In particular, angiographic magnetic resonance image data sets are presented to an expert diagnostician for the creation of a diagnosis, most often in a maximum intensity projection over all acquired slices in different spatial directions.

Therefore, in accordance with the invention, a coding of the magnetic resonance signals in the slice direction is foregone by the use of projection acquisition sequences. The magnetic resonance image data thus can be created very quickly by means of the projection acquisition sequences. This can lead to a marked shortening of the measurement time to acquire the angiographic magnetic resonance image data sets. An improved time resolution of the depiction of the contrast agent distribution thus can be achieved and/or the comfort for the examined person can be increased. If a reduction of the measurement time is not absolutely necessary, the projection acquisition sequences can be implemented with an increased resolution, for example. Furthermore, the projection acquisition sequences according to the invention enable slow gradient switchings to be used, which can be executed at magnetic resonance apparatuses with less powerful gradient systems. The projection acquisition sequences advantageously lead to a reduced noise volume, and thus to an increased comfort for the examined person during the acquisition of the magnetic resonance image data. All of these advantages can be achieved without an appreciable loss of image quality.

In an embodiment, the first projection acquisition sequence and/or the second projection acquisition sequence has an echo time of at most 1 ms. In particular, the first projection acquisition sequence and/or the second projection acquisition sequence includes an echo time of at most 500 μs, preferably at most 250 advantageously at most 100 μs and most advantageously at most 70 μs. The first and/or second projection acquisition sequence can thus include ultrashort echo times and/or a magnetic resonance sequence with ultrashort echo times. The use of echo times of at most 1 ms or shorter is inasmuch advantageous with regard to the proposed method since a dephasing of the spins (in particular necessitated by a use of a projection acquisition sequence) along the projection direction can therefore be avoided. Furthermore, by the use of ultrashort echo times, a dephasing of the spins along the projection direction is typically no longer of such significant consequence. The use of short echo times thus leads to an improvement of the image quality of the at least one magnetic resonance image data set. The combination of the proposed projection acquisition sequences with ultrashort echo times has thus turned out to be particularly advantageous.

In another embodiment, the first projection acquisition sequence and/or the second projection acquisition sequence includes a keyhole imaging sequence, which includes a separate acquisition of a center and an outer region of k-space. The outer region of k-space is preferably sampled (filled with data) radially. The center of k-space is preferably sampled in a Cartesian coordinate system and/or sampled in a single point. The magnetic resonance image data acquired in the center of k-space are typically used for an improvement of the contrast of the magnetic resonance image data sets. The magnetic resonance image data acquired in the outer region of k-space are typically used for an improvement of the resolution of the magnetic resonance image data set. Given the acquisition of the second magnetic resonance image data by means of projection acquisition sequences, the center of k-space is advantageously sampled first, and only after this is the outer region of k-space sampled. The reason for this is that the acquisition of the second magnetic resonance image data is for the most part time-critical after the introduction of the contrast agent and places fewer demands on the resolution of the projection acquisition sequences. The use of the keyhole imaging sequence is a particularly advantageous method to implement the projection acquisition sequences, in particular given the use of very short echo times. The proposed keyhole imaging sequence is also very quiet, and therefore offers a great comfort to the examined person.

In another embodiment, the acquisition of the first magnetic resonance image data is implemented using multiple first projection acquisition sequences, wherein the multiple first projection acquisition sequences differ in their projection directions. The multiple first projection acquisition sequences typically differ in alternation in their projection directions. Up to a maximum of 128 projection acquisition sequences with 128 different projection directions can be implemented. Typically, a maximum of 16 projection acquisition sequences with 16 different projection directions are implemented.

The implementation of at least four projection acquisition sequences with at least four projection directions is advantageous. The projection direction is typically that direction in which the projection is implemented. The projection direction is that direction along which the projection acquisition sequence automatically already averages the acquired magnetic resonance signals during the acquisition of the magnetic resonance image data. The acquisition of the second magnetic resonance image data can similarly be implemented using multiple second projection acquisition sequences, wherein the multiple second projection acquisition sequences differ in their projection directions. The first projection directions then advantageously coincide with the second projection directions. Multiple magnetic resonance image data sets can then particularly simply be created with different projection directions. Multiple magnetic resonance image data sets and/or advantageously a three-dimensional magnetic resonance image data set can be created from the magnetic resonance image data acquired by multiple projection acquisition sequences with different projection directions. The three-dimensional magnetic resonance image data set is thereby advantageously tailored to the requirements of an expert personnel performing the diagnosis. Multiple projection directions also enable a particularly simple assessment of the vessels of the examined person along different spatial directions by an expert personnel performing the diagnosis.

In a further embodiment, the acquisition of the first magnetic resonance image data is implemented using multiple first projection acquisition sequences, wherein the multiple first projection acquisition sequences are executed using different coil channels of an RF reception coil of the magnetic resonance apparatus. A coil channel (also called a coil element) of a reception coil or radio-frequency coil typically represents a spatially delimited acquisition unit of the radio-frequency coil. A coil channel can substantially independently receive magnetic resonance signals. For example, a coil channel of a radio-frequency coil can include a conductor loop of the reception coil. A radio-frequency coil typically comprises multiple coil channels, possibly up to 256, typically between 4 and 32. The use of multiple coil channels offers the advantage that multiple projection acquisition sequences can be implemented simultaneously with different coil channels of the radio-frequency coil. A reduction of the measurement time to acquire the magnetic resonance image data can therefore again can be achieved since multiple projection acquisition sequences can be implemented simultaneously, possibly with different projection directions. The use of different coil channels of the reception coil also enables the fact that the first projection acquisition sequences and/or the second projection acquisition sequences have a limited projection thickness. The acquisition of the second magnetic resonance image data can similarly be implemented using multiple second projection acquisition sequences, wherein the multiple second projection acquisition sequences are executed using different coil channels of a reception coil of the magnetic resonance apparatus. In particular, the first coil channels then coincide with the second coil channels.

In another embodiment, the first projection acquisition sequence and/or the second projection acquisition sequence have a limited projection thickness. In the normal case, projection acquisition sequences have a projection thickness going to infinity due to the omission of a coding in the slice direction. Typically, the projection thickness is that path length in the projection direction, over which the magnetic resonance signals are averaged upon acquisition of magnetic resonance image data by means of projection acquisition sequences. The projection thickness can be limited to 1 m, advantageously to 50 cm, most advantageously to 30 cm. If only individual coil channels or even a single coil channel of a reception coil which has a limited signal penetration depth and/or a limited signal reception profile are used to acquire magnetic resonance signals, the first projection acquisition sequence and/or the second projection acquisition sequence can have a limited projection thickness. The reason for this is that the coil channel receives only a portion of the magnetic resonance signals in the examination region due to the limited signal reception profile. A limited projection thickness of the projection acquisition sequence is then achieved without the use of time-consuming slice coding gradients. In a magnetic resonance image data set, preferably only one projection of a portion of the examination subject (for example only of a leg given a sagittal acquisition) is thereby shown. This can facilitate the assessment of the magnetic resonance image data sets by an expert personnel.

The image data acquisition unit according to the invention has a computer which is designed to execute a method according to the invention. The image data acquisition unit according to the invention is thus fashioned to execute a method to create at least one magnetic resonance image data set of an examination subject by means of a magnetic resonance apparatus. The image data acquisition unit is designed to acquire first magnetic resonance image data using a first projection acquisition sequence. The image data acquisition unit is furthermore designed to execute an acquisition of second magnetic resonance image data after administration of contrast agent using a second projection acquisition sequence. The computer of the image data acquisition unit is designed to execute a creation of at least one magnetic resonance image data set using the first magnetic resonance image data and the second magnetic resonance image data. The image data acquisition unit can optionally have an introduction device—in particular an injector—which is designed to introduce a contrast agent into the examination subject after the acquisition of the first magnetic resonance image data.

In an embodiment, the image data acquisition unit is designed such that the first projection acquisition sequence and/or the second projection acquisition sequence has an echo time of at most 1 ms.

In another embodiment, the image data acquisition unit is designed such that the first projection acquisition sequence and/or the second projection acquisition sequence includes a keyhole imaging sequence which includes a separate acquisition of a center and an outer region of k-space.

In another embodiment, the image data acquisition unit is designed such that the acquisition of the first magnetic resonance image data is implemented using multiple first projection acquisition sequences, wherein the multiple first projection acquisition sequences differ in their projection directions.

In another embodiment, the image data acquisition unit is designed such that the acquisition of the first magnetic resonance image data is implemented using multiple first projection acquisition sequences, wherein the multiple first projection acquisition sequences are executed using different coil channels of a reception coil of the magnetic resonance apparatus.

In another embodiment, the image data acquisition unit is designed such that the first projection acquisition sequence and/or the second projection acquisition sequence has a limited projection thickness.

The image data acquisition unit can have additional control components that are necessary and/or advantageous for execution of a method according to the invention. The image data acquisition unit can also be designed to send control signals to the magnetic resonance apparatus and/or to receive and/or process control signals in order to execute a method according to the invention. For this purpose, computer programs and additional software can be stored in a memory unit of the image data acquisition unit, by means of which computer programs and additional software a processor of the image data acquisition unit automatically controls and/or executes a method workflow of a method according to the invention. The image data acquisition unit can therefore create angiographic magnetic resonance image data sets with a shortened measurement time, reduced noise volume and lower gradient power.

The magnetic resonance apparatus according to the invention has such an image data acquisition unit. The magnetic resonance apparatus according to the invention is therefore designed to execute a method according to the invention with the image data acquisition unit. The image data acquisition unit can be integrated into the magnetic resonance apparatus. The image data acquisition unit can also be installed separate from the magnetic resonance apparatus. The image data acquisition unit can be connected with the magnetic resonance apparatus. Embodiments of the magnetic resonance apparatus according to the invention are designed analogous to the embodiments of the method according to the invention. The magnetic resonance apparatus can therefore create magnetic resonance image data sets with a shortened measurement time, reduced noise volume and lower gradient power.

The present invention also encompasses a non-transitory, computer-readable data storage medium encoded with programming instructions that, when the storage medium is loaded into a programmable computer of a magnetic resonance apparatus, cause the magnetic resonance apparatus to execute the method according to the invention as described above, in all embodiments.

The computer must have the requirements (for example a working memory, a graphics card or a logic unit) so that the respective method steps can be executed efficiently. The storage medium is loaded into the processor of a local computer that can be directly connected with the magnetic resonance apparatus or is designed as part of the magnetic resonance apparatus. Examples of such an electronically readable data medium are a DVD, a magnetic tape or a USB stick on which is stored electronically readable control information, in particular software. All embodiments according to the invention of the method described above can be implemented when this control information (software) is read from the data medium and stored in a controller and/or computer of a magnetic resonance apparatus.

### BRIEF DESCRIPTION OF THE DRAWINGS

FIG. 1 shows a magnetic resonance apparatus according to the invention for execution of a method according to the invention, in a schematic illustration.

FIG. 2 is a flowchart of an embodiment of the method according to the invention.

FIG. 3 illustrates a measurement configuration to create angiographic magnetic resonance image data sets of a leg region of an examined person.

### DESCRIPTION OF THE PREFERRED EMBODIMENTS

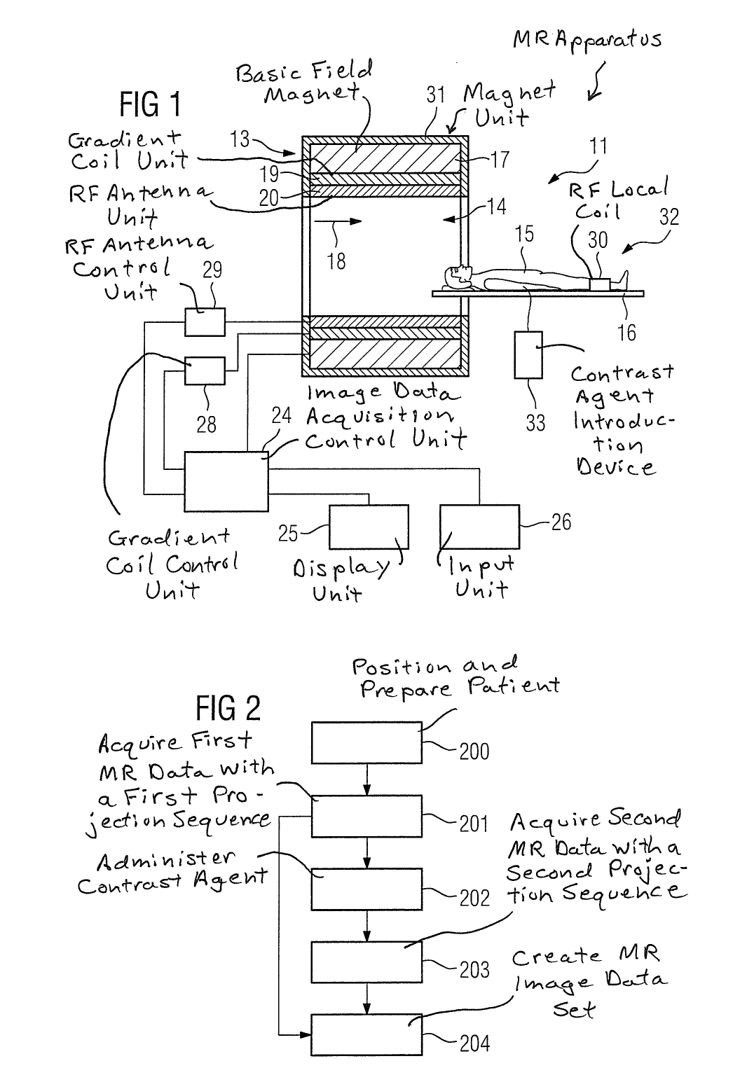

FIG. 1 schematically shows a magnetic resonance (MR) apparatus 11 according to the invention for execution of a method according to the invention. The magnetic resonance apparatus 11 has a scanner (formed by a magnet unit 13) with a basic field magnet 17 to generate a strong and in particular constant basic magnetic field 18. In addition to this, the magnetic resonance apparatus 11 has a cylindrical acquisition region 14 for introduction of an examined person 15, wherein the acquisition region 14 is cylindrically enclosed by the magnet unit 13 in a circumferential direction. The examined person 15 can be slid into the acquisition region 14 by a support device 16 of the magnetic resonance apparatus 11. For this purpose, the support device 16 has a bed table that is arranged so as to be movable within the magnetic resonance apparatus 11. The magnet unit 13 is externally shielded by a housing casing 31 of the magnetic resonance apparatus.

Furthermore, the magnet unit 13 has a gradient coil unit 19 to generate magnetic field gradients that are used for a spatial coding during an imaging. The gradient coil unit 19 is controlled by a gradient coil control unit 28. Furthermore, the magnet unit 13 has a radio-frequency (RF) antenna unit 20 (which, in the shown case, is designed so as to be permanently integrated into the magnetic resonance apparatus 10) and a radio-frequency antenna control unit 29 for an excitation of nuclear spins to deflect them from a polarization that arises in the basic magnetic field 18 generated by the basic magnet 17. The radio-frequency antenna unit 20 is controlled by the radio-frequency antenna control unit 29 and radiates radio-frequency pulses into an examination space that is essentially formed by the acquisition region 14.

The magnetic resonance apparatus 11 has an image data acquisition control unit 24 to control the basic field magnet 17, the gradient coil unit 28 and the radio-frequency antenna control unit 29. The image data acquisition unit 24 centrally controls the magnetic resonance apparatus 11, for example the implementation of magnetic resonance sequences. Control information (for example imaging parameters) as well as reconstructed magnetic resonance images can be displayed on a display unit 25, for example on at least one monitor of the magnetic resonance apparatus 11 for an operator. In addition to this, the magnetic resonance apparatus 11 has an input unit 26 via which information and/or imaging parameters can be entered by an operator during a measurement process. The image data acquisition unit 24 can include the gradient control unit 28 and/or radio-frequency antenna control unit 29 and/or the display unit 25 and/or the input unit 26. The image data acquisition unit has a computer (not further shown) of the image data acquisition unit 24.

Furthermore, the magnetic resonance apparatus 11 (in particular the image data acquisition unit 24) has an introduction device 33, in particular an injector 33 which is designed to introduce (in particular to inject) a contrast agent into the examined person 15, in the shown case into an arm vein of the examined person 15.

Furthermore, the magnetic resonance apparatus 11 has a radio-frequency coil 30 that is designed to acquire magnetic resonance image data. The radio-frequency coil 30 is positioned for a magnetic resonance examination by a medical operating personnel at a body region of the examined person 15 that is to be examined. In this case, the body region of the examined person 15 that is to be examined is the leg region 32 of the examined person 15. In the present exemplary embodiment, the radio-frequency coil 30 is formed by a body antenna unit. The radio-frequency coil 30 may alternatively be a knee antenna unit and/or dorsal antenna unit, etc.

The shown magnetic resonance apparatus 11 can naturally include additional components that magnetic resonance apparatuses 11 conventionally have. The general functioning of a magnetic resonance apparatus 11 is known to those skilled in the art, such that a detailed description of the additional components is not necessary.

FIG. 2 shows a flowchart of an embodiment of the method according to the invention. In a first method step 200, the examination subject 15 (in particular the examined person 15) is positioned on the support device 16 in the acquisition region 14 of the magnetic resonance apparatus 11 to acquire at least one angiographic magnetic resonance image data set. Furthermore, the examination subject 16 is prepared for the measurement in that—for example—a radio-frequency local coil 30 is positioned on an examination region, for example the leg region 32 of the examined person 15.

In a further method step 201, an acquisition of first magnetic resonance image data takes place using a first projection acquisition sequence. For this, multiple keyhole imaging sequences with a very short echo time of 70 μs are started by the image data acquisition unit 24 in projection mode, wherein the multiple keyhole imaging sequences differ in their projection directions. The imaging parameters of the projection acquisition sequences are passed by the image data acquisition unit 24 to the gradient control unit 28 and the radio-frequency antenna control unit 29. Control commands which are used to control the gradient coil unit 29 and the radio-frequency antenna unit 20 are then generated from the imaging parameters in the gradient control unit 28 and radio-frequency antenna control unit 29. Naturally, other projection acquisition sequences can also be used in addition to keyhole imaging sequences. Although the use of a short echo time of 70 μs is very advantageous, it is not absolutely necessary.

In a further method step 202, a contrast agent is injected into the bloodstream of the examined person 15 by means of the introduction device 33 (the injector 33).

In a further method step 203, second magnetic resonance image data are acquired by means of the image data acquisition unit 24 using a second projection acquisition sequence. The sequence hereby proceeds analogous to the additional method step 201 (the acquisition of the first magnetic resonance image data). The acquisition of the second magnetic resonance image data starts shortly after the introduction of the contrast agent. The center of k-space is initially sampled by a keyhole imaging sequence in order to generate the necessary contrast for the time-critical acquisition of the distribution of the contrast agent. Although this procedure is naturally advantageous, but not absolutely necessary. Imaging parameters—for example the keyhole imaging sequence that is used, the number and the alignment of the projection directions, the field of view, the echo time and the repetition time—are kept constant between the acquisition of the first magnetic resonance image data and the acquisition of the second magnetic resonance image data.

In a further method step 204, the first magnetic resonance image data and the second magnetic resonance image data are offset with one another using a weighted subtraction by means of the computer of the image data acquisition unit 24. This leads to a creation of multiple angiographic magnetic resonance image data sets with multiple projection directions. The number of angiography magnetic resonance image data sets thereby corresponds to the number of projection directions during the acquisition of the first magnetic resonance image data or the second magnetic resonance image data.

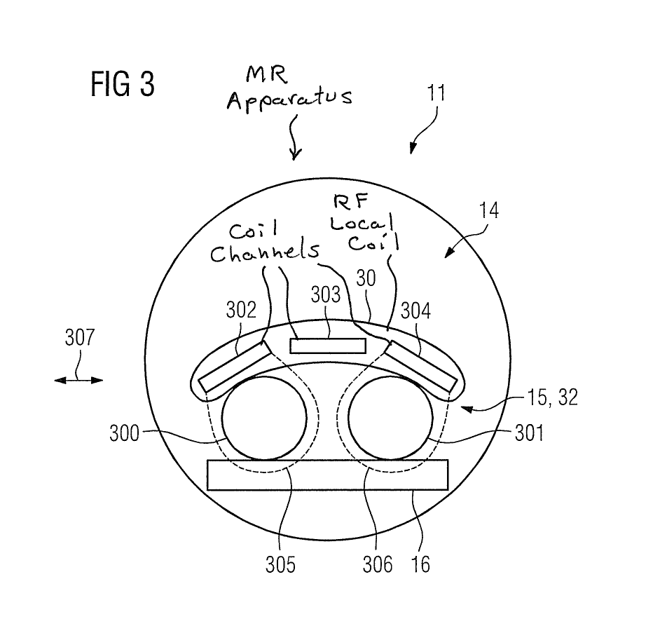

FIG. 3 shows a measurement configuration to create angiography magnetic resonance image data sets of a leg region 32 of an examined person 15. The examined person 15 is positioned on the back within the acquisition region 14 of the magnetic resonance apparatus 11 on the bearing device 16. A radio-frequency coil 30 (in this case a body antenna unit) is positioned on the leg region 32 of the examined person 15, in particular the right leg 30 and the left leg 301 of the examined person 15.

The radio-frequency local coil 30 comprises a right coil channel 302, a middle coil channel 303 and a left coil channel 304. The right coil channel 302 is positioned in the region of the right leg 300 of the examined person 15 and has a right signal reception profile 305. The right signal reception profile 305 characterizes the region from which the right coil channel 302 can receive magnetic resonance signals. In the shown cross section, the right signal reception profile 305 encompasses the entire right leg 300 of the examined person 15. The right signal reception profile 305 excludes the left leg 301 of the examined person 15. The left coil channel 304 is positioned in the region of the left leg 301 of the examined person 15 and has a left signal reception profile 306. The left signal reception profile 306 thereby characterizes the region from which the left coil channel 304 can receive magnetic resonance signals. In the shown cross section, the left signal reception profile 306 includes the entire left leg 301 of the examined person 15. The left signal reception profile 306 excludes the right leg 300 of the examined person 15.

During the additional method steps 201 and 203, two projection acquisition sequences (a left projection acquisition sequence and a right projection acquisition sequence) are respectively implemented by means of the image data acquisition unit 24.

The left and right projection acquisition sequences respectively have a sagittal projection direction 307 which, in FIG. 3, is indicated by a double arrow. The sagittal projection direction 307 is arranged orthogonally to a longitudinal extent of the examined person 15 on the bearing device 16. The sagittal projection direction 307 proceeds through both legs 300, 301 of the examined person 15. For a right projection acquisition sequence with the sagittal projection direction 307, the magnetic resonance signals are acquired by means of the right coil channel 302 with the right signal reception profile 305. For a left projection acquisition sequence with the sagittal projection direction 307, magnetic resonance signals are acquired by means of the left coil channel 304 with the left signal reception profile 306.

Since projection acquisition sequences fundamentally forego a coding in the slice direction, projection acquisition sequences typically have a projection thickness going to infinity. Without the use of different coil channels 302, 304 for the projection acquisition sequences, the projection thickness going to infinity would lead to the situation that magnetic resonance signals of both legs 300, 301 would be acquired for the left and right projection acquisition sequences along the sagittal projection direction 307. This would lead to the situation that both legs 300, 301 (in particular the vessels of both legs 300, 301) would be shown superimposed in the magnetic resonance image data sets created from the projection acquisition sequences with the projection thickness going to infinity. This would hinder a diagnosis of the vessels of both legs 300, 301 for an expert person since the blood vessels of both legs 300, 301 would be hard to differentiate from one another in the magnetic resonance image data sets.

However, because only the right coil channel 302 with the right signal reception profile 305 is used for acquisition of the magnetic resonance signals for the right projection acquisition sequence, during the right projection acquisition sequence a projection acquires only the right leg 300 of the examined person 15 in a sagittal projection direction 307. The reason for this is the right signal reception profile 305 acquires only magnetic resonance signals from the right leg 300 of the examined person 15. The effective projection thickness of the right projection acquisition sequence therefore corresponds to the diameter of the right leg 300. In that only the left coil channel 304 with the left signal reception profile 306 is used to acquire the magnetic resonance signals for the left projection acquisition sequence, a projection of only the left leg 301 of the examined person 15 is similarly acquired in a sagittal projection direction 307 during the left projection acquisition sequence. The reason for this is that the left signal reception profile 306 acquires magnetic resonance signals only from the left leg 301 of the examined person 15. The effective projection thickness of the left projection acquisition sequence therefore corresponds to the diameter of the left leg.

In the further method step 204, the left projection acquisition sequence and right projection acquisition sequence that are implemented during the acquisition of the first magnetic resonance image data are offset with the left projection acquisition sequence and right projection acquisition sequence implemented during the acquisition of the second magnetic resonance image data. From these, two magnetic resonance image data sets are created, of which one shows a projection through the blood vessels of the right leg 300 of the examined person 15 and one shows a projection of the blood vessels of the left leg 301. For an expert diagnostician, a simplified assessment of the vessels separately for both legs 300, 301 is now possible.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to create at least one magnetic resonance image data set of an examination subject, comprising:
operating a magnetic resonance data acquisition unit, in which an examination subject is situated, to acquire first magnetic resonance image data from the subject, using a first projection acquisition sequence;
operating said magnetic resonance data acquisition unit to acquire second magnetic resonance image data from the examination subject, after administering a contrast agent to the subject, using a second projection acquisition sequence; and
providing said first magnetic resonance image data and said second magnetic resonance image data to a computerized processor and, in said processor, automatically creating at least one magnetic resonance image data set using the first magnetic resonance image data and the second magnetic resonance image data, and making said at least one magnetic resonance image data set available at an output of said processor in electronic form, as a data file.

2. A method as claimed in claim 1 comprising operating said magnetic resonance data acquisition unit to execute said first projection acquisition sequence with an echo time of at most 1 ms.

3. A method as claimed in claim 1 comprising operating said magnetic resonance data acquisition unit to execute said second projection acquisition sequence with an echo time of at most 1 ms.

4. A method as claimed in claim 1 comprising operating said magnetic resonance data acquisition unit to execute each of said first projection acquisition sequence and said second projection acquisition sequence with an echo time of at most 1 ms.

5. A method as claimed in claim 1 comprising operating said magnetic resonance data acquisition unit in said first projection acquisition sequence to enter said first magnetic resonance image data into an electronic memory organized as k-space, according to a keyhole imaging sequence wherein said first magnetic resonance image data are separately entered into a center region of k-space and into an outer region of k-space surrounding said center region.

6. A method as claimed in claim 1 comprising operating said magnetic resonance data acquisition unit in said second projection acquisition sequence to enter said second magnetic resonance image data into an electronic memory organized as k-space, according to a keyhole imaging sequence wherein said second magnetic resonance image data are separately entered into a center region of k-space and into an outer region of k-space surrounding said center region.

7. A method as claimed in claim 1 comprising operating said magnetic resonance data acquisition unit in said first projection acquisition sequence to enter said first magnetic resonance image data into an electronic memory organized as k-space, according to a keyhole imaging sequence wherein said first magnetic resonance image data are separately entered into a center region of k-space and into an outer region of k-space surrounding said center region, and operating said magnetic resonance data acquisition unit in said second projection acquisition sequence to enter said second magnetic resonance image data into an electronic memory organized as k-space, according to a keyhole imaging sequence wherein said second magnetic resonance image data are separately entered into a center region of k-space and into an outer region of k-space surrounding said center region.

8. A method as claimed in claim 1 comprising operating said magnetic resonance data acquisition unit to acquire said first magnetic resonance image data by executing multiple first projection acquisition sequences, respectively differing from each other in projection directions.

9. A method as claimed in claim 1 comprising operating said magnetic resonance data acquisition unit to acquire said first magnetic resonance image data by executing multiple first projection acquisition sequences respectively using different coil channels of a reception coil of said magnetic resonance data acquisition unit.

10. A method as claimed in claim 1 comprising operating said magnetic resonance data acquisition unit to execute said first projection acquisition sequence with a limited projection thickness.

11. A method as claimed in claim 1 comprising operating said magnetic resonance data acquisition unit to execute said second projection acquisition sequence with a limited projection thickness.

12. A method as claimed in claim 1 comprising operating said magnetic resonance data acquisition unit to execute each of said first projection acquisition sequence and said second projection acquisition sequence with a limited projection thickness.

13. A magnetic resonance apparatus comprising:
a magnetic resonance data acquisition unit in which an examination subject is situated;
a control unit configured to operate said magnetic resonance data acquisition unit to acquire first magnetic resonance image data from the subject, using a first projection acquisition sequence;
a contrast agent introduction unit configured to administer contrast agent to the examination subject after acquisition of said first magnetic resonance image data;
said control unit being configured to operate said magnetic resonance data acquisition unit to acquire second magnetic resonance image data from the examination subject, after administering a contrast agent to the subject, using a second projection acquisition sequence; and
a computerized processor provided with said first magnetic resonance image data and said second magnetic resonance image data, said processor being configured to automatically create at least one magnetic resonance image data set using the first magnetic resonance image data and the second magnetic resonance image data, and to make said at least one magnetic resonance image data set available at an output of said processor in electronic form, as a data file.

14. A non-transitory, computer-readable data storage medium encoded with programming instructions, said storage medium being loaded into a control computer of a magnetic resonance apparatus comprising a magnetic resonance data acquisition unit, said programming instructions causing said control computer to:
operate said a magnetic resonance data acquisition unit, in which an examination subject is situated, to acquire first magnetic resonance image data from the subject, using a first projection acquisition sequence;
operate said magnetic resonance data acquisition unit to acquire second magnetic resonance image data from the examination subject, after administering a contrast agent to the subject, using a second projection acquisition sequence; and
create at least one magnetic resonance image data set using the first magnetic resonance image data and the second magnetic resonance image data, and make said at least one magnetic resonance image data set available at an output of said processor in electronic form, as a data file.
